(19) United States
(12) Patent Application Publication
(10) Pub. No.: US 2022/0226388 A1
(43) Pub. Date: Jul. 21, 2022

(54) CELL CULTURE FOR TREATING INFLAMMATORY DISEASE

(71) Applicant: TERUMO KABUSHIKI KAISHA, Tokyo (JP)

(72) Inventors: Fumiya OHASHI, Kanagawa (JP); Masaki MATSUMURA, Kanagawa (JP)

(21) Appl. No.: 17/716,269

(22) Filed: Apr. 8, 2022

Related U.S. Application Data
(63) Continuation of application No. PCT/JP2020/039014, filed on Oct. 16, 2020.

(30) Foreign Application Priority Data
Oct. 17, 2019 (JP) 2019-190479

(51) Int. Cl. A61K 35/34; C12N 5/077; A61P 1/12

(52) U.S. Cl. CPC A61K 35/34; C12N 5/0658; C12N 2539/10; C12N 2513/00; A61P 1/12

(57) ABSTRACT

Systems and methods of the present disclosure include a cell culture, a method for producing the cell culture, a kit for producing the cell culture, a method for treating inflammatory disease using the cell culture, and the like, for treating inflammatory disease. The cell culture containing cells can be derived from skeletal muscle.

### CROSS REFERENCE TO RELATED APPLICATIONS

The present application is a continuation of and claims benefit to PCT Application No. PCT/JP2020/039014 filed on Oct. 16, 2020, entitled “CELL CULTURE FOR TREATING INFLAMMATORY DISEASE” which claims priority to Japanese Application No. JP2019-190479 filed on Oct. 17, 2019. The entire disclosures of the applications listed above are hereby incorporated herein by reference, in their entirety, for all that they teach and for all purposes.

### FIELD

The present disclosure relates to a cell culture containing cells derived from skeletal muscle, a method for producing the cell culture, a kit for producing the cell culture, and a method for treating inflammatory disease using the cell culture, and the like, for treating inflammatory disease.

### BACKGROUND

In recent years, to repair damaged tissues or the like, attempts to transplant various cells have been made. For example, for repairing myocardial tissues damaged due to an ischemic heart disease such as angina pectoris or myocardial infarction, attempts have been made to utilize fetal cardiomyocytes, myoblast cells, mesenchymal stem cells, cardiac stem cells, embryonic stem (ES) cells, induced pluripotent stem (iPS) cells, and the like (See Haraguchi et al., Stem Cells Transl Med. 2012 February; 1(2); 136-41 (“Haraguchi”)).

As an example of such attempts, sheet-shaped cell cultures, which are obtained by forming cells into a sheet shape, have been developed, and some of the sheet-shaped cell cultures are in the stage of clinical application. Examples of such clinical application include a sheet-shaped cell culture, which can be attaching to the outside of a treatment site in order to prevent perforation or the like after endoscopic submucosal dissection (See Japanese Patent Application No. 2018-140977), and a sheet-shaped cell culture containing adipocytes, which improves cardiac function in a patient with heart disease by secreting adiponectin (See JP 5661048 B).

Inflammatory bowel disease is an intractable disease of which the cause of the onset has not been clearly understood, and includes mainly Crohn disease, and ulcerative colitis. It is estimated that there are more than 220,000 patients in total with Crohn disease or ulcerative colitis in Japan. As the method for treating such a disease, at present, for example, administration of a steroid drug, an immunosuppressive agent, an anti-TNF-α antibody preparation, or the like has been performed.

As the new therapeutic method for inflammatory bowel disease, a method of administering mesenchymal stem cells having various actions such as tissue differentiation, angiogenesis, and regulation of immune function has been proposed. For example, a method of administering intravenously or intralymphatically mesenchymal stem cells or adipose stem cells to an animal model of enteritis or chronic rheumatoid arthritis (See JP 2017-35094 A, Japanese Patent Application No. 2009-7321, and JP 6512759 B), a method of administering intestinally or intravenously a culture supernatant of mesenchymal stem cells to a rat model of enteritis (See JP 2017-137268 and JP 6132459 B), a method of regulating peripheral immune response by mesenchymal stem cells into which genes have been transferred (See JP 2019-6790), and the like are known.

### SUMMARY

An object of the present disclosure is to provide a cell culture containing cells derived from skeletal muscle, a method for producing the cell culture, a kit for producing the cell culture, a method for treating inflammatory disease using the cell culture, and the like, for treating inflammatory disease.

In the treatment of inflammatory bowel disease using mesenchymal stem cells, there remains a problem that it is difficult to culture mesenchymal stem cells suitable for a subject to be administered in an amount that can be administered as a drug, the bioadhesiveness at a desired site is insufficient when mesenchymal stem cells are administered intravenously or intralymphatically, and the like.

The present inventors have found that a culture supernatant of a sheet containing cells derived from skeletal muscle contains a component useful for treatment of inflammatory bowel disease, such as myokine, and as a result of further studies based on such a finding, the present inventors have completed the present invention.

Embodiments of the present disclosure relate to the following: a cell culture for treating inflammatory disease, including cells derived from skeletal muscle.

Any of the features herein, wherein the cell culture is a sheet-shaped cell culture.

Any of the features herein, wherein the cell culture secretes myokine.

Any of the features herein, wherein the inflammatory disease is generated in the lower gastrointestinal tract.

Any of the features herein, wherein the inflammatory disease is inflammatory bowel disease.

Any of the features herein, wherein the cell culture is used for transplantation to the serosal side of the gastrointestinal tract.

Any of the features herein, wherein a method for producing the sheet-shaped cell culture includes: seeding cells on a substrate; forming the seeded cells into a sheet-shaped cell culture; and detaching the formed sheet-shaped cell culture from the substrate.

Any of the features herein, wherein a kit for producing the cell culture includes: cells; and a culture medium and a substrate, for culturing the cells.

Any of the features herein, wherein a method for treating inflammatory disease includes transplanting a cell culture containing cells derived from skeletal muscle.

Any of the features herein, wherein the cell culture is a sheet-shaped cell culture.

Any of the features herein, wherein the cell culture secretes myokine.

Any of the features herein, wherein the inflammatory disease is generated in the lower gastrointestinal tract.

Any of the features herein, wherein the inflammatory disease is inflammatory bowel disease.

Any of the features herein, wherein transplantation of the cell culture is transplantation to the serosal side of the gastrointestinal tract.

According to embodiments of the present disclosure, by performing transplantation of a cell culture containing cells derived from skeletal muscle, treatment of inflammatory disease can be promoted.

### BRIEF DESCRIPTION OF THE DRAWINGS

FIG. 1 shows amounts of various cytokines contained in a culture supernatant of a sheet-shaped cell culture containing human myoblast cells, which are measured by using a Bio-Plex™ multisystem according to embodiments of the present disclosure;

FIGS. 2A-2B shows a plan for a transplant test in a mouse model of colitis where FIG. 2A shows a follow-up group, and FIG. 2B shows a test group, where two percent dextran sulfate sodium (DSS) is administered in free drinking water in both of the groups from the start date of the test to the 4th day, where the thin arrow in FIG. 2B indicates the day when a sheet-shaped cell culture was transplanted, and where the thick arrows indicate the days when the autopsy was performed according to embodiments of the present disclosure;

FIGS. 3A-3D show photographs of tissues collected from mice in a sheet-shaped cell culture transplantation group and in a sham surgery group by autopsy on the 31st day after the start of the test, where FIG. 3A is a photograph of the lower gastrointestinal tract taken out from a mouse in the sheet-shaped cell culture transplantation group; where FIG. 3B is a photograph of the inner membrane of the gastrointestinal tract confirmed by cutting open the lower gastrointestinal tract, where FIG. 3C is a photograph of the lower gastrointestinal tract taken out from a mouse in the sham surgery group, and where FIG. 3D is a photograph of the inner membrane of the gastrointestinal tract confirmed by cutting open the lower gastrointestinal tract according to embodiments of the present disclosure;

FIGS. 4A-4D show images of colon tissues of mice in the sheet-shaped cell culture transplantation group and in the sham surgery group, obtained by autopsy on the 31st day after the start of the test, where FIG. 4A shows an image of a cross section of a mouse in the sheet-shaped cell culture transplantation group obtained by autopsy on the 31st day after the start of the test, where FIG. 4B is an enlarged image of a portion surrounded by a dashed line in FIG. 4A, where FIG. 4C shows an image of a cross section of a mouse in the sham surgery group obtained by autopsy on the 31st day after the start of the test, and where FIG. 4D is an enlarged image of a portion surrounded by a dashed line in FIG. 4C according to embodiments of the present disclosure;

FIG. 5 is a graph showing changes in the body weight of the sheet-shaped cell culture transplantation group and the sham surgery group where the squares indicate the sham surgery group, where the black circles indicate the sheet-shaped cell culture transplantation group, where the vertical axis shows the body weight of a mouse, where the horizontal axis shows the number of days elapsed after the start of the test, and where, on the 11th day after the start of the test, transplantation of a sheet-shaped cell culture was performed according to embodiments of the present disclosure;

FIG. 6 is a graph showing changes in the body weight of the sheet-shaped cell culture transplantation group and the sham surgery group, using the body weight of each mouse on the 11th day after the start of the test as the basis, where the squares indicate the sham surgery group, where the black circles indicate the sheet-shaped cell culture transplantation group, where the vertical axis shows the changes in the body weight from the basis, and where the horizontal axis shows the number of days elapsed after the start of the test according to embodiments of the present disclosure;

FIG. 7 is a graph showing inflammation scores of mice in the sheet-shaped cell culture transplantation group and in the sham surgery group, where the vertical axis shows the value of inflammation score (DAI), where the horizontal item axis shows the date when the score was measured, where the squares indicate the sham surgery group, and where the black circles indicate the sheet-shaped cell culture transplantation group according to embodiments of the present disclosure; and

FIG. 8 shows a table that shows histopathological evaluation of tissues of mice in respective groups according to embodiments of the present disclosure.

### DETAILED DESCRIPTION

Hereinafter, the present disclosure will be described in detail.

One feature of the present disclosure relates to a cell culture containing cells derived from skeletal muscle, for treating inflammatory disease.

In the present disclosure, the inflammatory disease is a disease characterized by symptoms of inflammation. Examples of the inflammatory disease include, but are not limited to, a systemic or local inflammation disease such as an allergy, or an immune complex disease, a gastrointestinal disease such as Crohn disease, ulcerative, acute or ischemic colitis, intestinal Behcet disease, diverticulitis, or cholecystitis, a skin-related disease such as dermatitis, a cardiovascular disease such as endocarditis, or myocarditis, a respiratory disease such as asthma, tuberculosis (TB), bronchiectasis, or chronic obstructive pulmonary disease (COPD), a connective tissue-related disease such as rheumatoid arthritis, osteomyelitis, or fasciitis, a urogenital system disease such as nephritis, a nervous system-related disease such as meningitis, encephalitis, or multiple sclerosis, a disease caused by infection with viruses, fungi, or microorganisms, an autoimmune disease such as thyroiditis, and a cancer.

In one embodiment, the inflammatory disease refers to a disease resulting from abnormal release of inflammatory cytokines. For example, the inflammatory disease in the present disclosure may refer to inflammatory bowel disease.

As used herein, the expression “cell culture” refers to a composition obtained through a cell culture step. In one embodiment, the cell culture of may be a sheet-shaped cell culture. As used herein, the expression “sheet-shaped cell culture” means a cell culture in a sheet shape formed by connecting cells to each other. The cells may be connected to each other directly (e.g., including connection through a cellular element such as an adhesion molecule) and/or through a mediator (e.g., interposed materials). The mediator is not particularly limited as long as it is a substance that can at least physically (e.g., mechanically) connect cells to each other, and the mediator, for example, may be an extracellular matrix or the like. The mediator is preferably derived from a cell, and particularly derived from a cell that forms a cell culture. The cells are at least physically (e.g., mechanically) connected to each other, but may be more functionally connected to each other (e.g., chemically, electrically, combinations thereof, and the like). The sheet-shaped cell culture may be formed of one cell layer (e.g., a single layer), or may also be formed of two or more cell layers, such as a laminated or multilayer body (e.g., a two-layer, three-layer, four-layer, five-layer, or six-layer sheet-shaped cell culture). Further, the sheet-shaped cell culture may have a three-dimensional structure having a thickness exceeding the thickness of one cell without showing any clear layered structure of the cells. For example, in the vertical section of the sheet-shaped cell culture, the cells may be non-uniformly (e.g., mosaic-like) arranged without being uniformly aligned in the horizontal direction.

The sheet-shaped cell culture preferably does not contain a scaffold (e.g., a support). The scaffold may be used in some cases in order to attach cells onto the surface of and/or to the inside of the scaffold and to maintain the physical integrity of the sheet-shaped cell culture. The scaffold, for example, may be made of a membrane comprising polyvinylidene difluoride (PVDF) or the like, but the sheet-shaped cell culture of the present disclosure can maintain the physical integrity even without such a scaffold. Further, in some embodiments of the present disclosure the sheet-shaped cell culture consists only of cell-derived substances forming the sheet-shaped cell culture, and does not contain any other substances.

In the present disclosure, the cell culture contains cells derived from skeletal muscle. In the present disclosure, the cells derived from skeletal muscle refer to satellite cells, myoblast cells, skeletal muscle cells, skeletal myotubes, and skeletal muscle fibers. Preferably, the cells derived from skeletal muscle are myoblast cells. The cells forming the cell culture of the present disclosure contain cells derived from skeletal muscle by 50% or more, 60% or more, 65% or more, 70% or more, 75% or more, 80% or more, 85% or more, or 90% or more, and preferably 60% or more.

The myoblast cells are well known in the technical field to which the present disclosure belongs, and can be prepared from skeletal muscle by any known method (e.g., method disclosed in JP 2007-89442 A, or the like). In some embodiments, commercially available myoblast cells may be obtained, such as catalog number: CC-2580 of Lonza Japan, product code 3520 of Cosmo Bio Co., Ltd., or the like. The myoblast cells are not limited to such cells, and can be identified by a marker such as CD56, α7 integrin, myosin heavy chain IIa, myosin heavy chain IIb, myosin heavy chain IId (IIx), MyoD, Myf5, Myf6, myogenin, desmin, or PAX3. In one embodiment, the myoblast cells are CD56 positive. In one embodiment, the myoblast cells are CD56 positive and desmin positive.

In embodiments where the myoblast cells are prepared from striated muscle tissue, the prepared cell population contains fibroblast cells. When the cell culture according to the present disclosure is produced, in a case where a cell population containing the myoblast cells prepared from striated muscle tissue is used, a certain amount of fibroblast cells is contained in the cell population. The fibroblast cells are well known in the technical field to which the present disclosure belongs, and can be identified by a marker such as TE-7 (see, e.g., Rosendaal et al., J Cell Sci. 1994, 107(Pt1): 29-37, Goodpaster et al. J Histochem Cytochem. 2008, 56(4): 347-358, and the like).

In one embodiment, the cells forming the cell culture of the present disclosure include the myoblast cells prepared from striated muscle tissue. Accordingly, the cell population used in production of the cell culture of the present disclosure can contain myoblast cells and fibroblast cells. In one embodiment, the cell population used in production of the cell culture of the present disclosure can have a CD56-positive rate of 50% or more, 60% or more, 65% or more, 70% or more, 75% or more, 80% or more, 85% or more, or 90% or more, and preferably 60% or more.

The cell population used in production of the cell culture of the present disclosure can contain fibroblast cells, and in a case where the content of the fibroblast cells is extremely high, the content of myoblast cells is lowered, and thus, this is not preferable. Accordingly, in one embodiment, the cell population used in production of the cell culture of the present disclosure can have a TE-7 positive rate of 50% or less, 40% or less, 35% or less, 30% or less, 25% or less, 20% or less, 15% or less, or 10% or less, and preferably 40% or less.

The cell population used in production of the cell culture of the present disclosure can contain cells other than the myoblast cells and the fibroblast cells. The higher the total value of the CD56-positive rate and the TE-7 positive rate is, the more preferable the cell population is, and the total value can be, for example, 80% or more, 85% or more, 90% or more, 95% or more, 96% or more, 97% or more, 98% or more, or 99% or more, and preferably 90% or more.

In the present disclosure, a cytokine is secreted from a cell culture containing cells derived from skeletal muscle.

In the present disclosure, the cytokine secreted from a cell culture containing cells derived from skeletal muscle is commonly referred to as a myokine, and examples of such a cytokine include, but are not limited to, IL-1b, IL-1ra, IL-2, IL-4, IL-5, IL-6, IL-7, IL-8, IL-9, IL-10, IL-12, IL-13, IL-15, IL-17, eotaxin, FGF-basic, G-CSF, GM-CSF, IFN-g, IP-10, MCP-1 (MCAF), MIP-1a, PDGF-bb, MIP-1b, RANTES, TNF-α, HGF-1, SDF-1, and VEGF.

In embodiments where the cell culture of the present disclosure is a sheet-shaped cell culture, the thickness of the sheet-shaped cell culture is not particularly limited. In embodiments where a single-layer sheet is used as the sheet-shaped cell culture, the thickness is usually a thickness of one or more cells, and varies depending on the kind of the cells forming the sheet-shaped cell culture, and in one embodiment, the sheet-shaped cell culture of the present disclosure has a thickness of 30 micrometers (μm) or more, and in one preferred embodiment, the sheet-shaped cell culture has a thickness of 50 μm or more. Examples of the range of the value of the sheet-shaped cell culture of the present disclosure include 30 μm to 200 μm, preferably 50 μm to 150 μm, and more preferably 60 μm to 100 μm. In a case where a laminated sheet is used as the sheet-shaped cell culture, the thickness does not exceed the value obtained by a thickness of the single-layer sheet multiplied by the number of laminated sheets. Accordingly, in embodiments where, for example, a sheet obtained by stacking five single-layer sheets in layers is used, the sheet has a thickness of 150 μm or more, and in one preferred embodiment, the sheet has a thickness of 250 μm or more. In such embodiments, examples of the range of the value of the sheet-shaped cell culture of the present disclosure include 150 μm to 1000 μm, preferably 250 μm to 750 μm, and more preferably 300 μm to 500 μm.

The cells composing the cell culture of the present disclosure can be derived from any organism that is to be subject to treatment with the cell culture, and examples of such an organism include, but are not limited to, a human, primates, a dog, a cat, a pig, a horse, a goat, a sheep, rodent animals (e.g., a mouse, a rat, a hamster, and a guinea pig), and a rabbit. Further, the cells composing the cell culture of the present disclosure may include only a single type of cell, or two or more kinds of cells. In a preferred embodiment of the present disclosure, two or more types of cells form the cell culture, and the content ratio (purity) of the most abundant cells is 60% or more, preferably 70% or more, and more preferably 75% or more, at the end of the cell culture production.

The cells may be cells derived from heterogeneous cells, or may also be cells derived from homogeneous cells. In this regard, when the cell culture is used for transplantation, the expression “cells derived from heterogeneous cells” means cells derived from an organism of a species different from that of the recipient. For example, in a case where the recipient is a human, cells derived from a monkey or a pig correspond to the cells derived from heterogeneous cells. Further, the expression “cells derived from homogeneous cells” means cells derived from an organism of the same species as that of the recipient. For example, in a case where the recipient is a human, human cells correspond to the cells derived from homogeneous cells. The cells derived from homogeneous cells include self-derived cells (also referred to as “self cells” or “autologous cells”), that is, recipient-derived cells, as well as cells derived from homogeneous non-self cells (also referred to as “non-autologous cells”). The self-derived cells are preferable in the present disclosure because the cells do not cause any rejection even when being transplanted. However, cells derived from heterogeneous cells, or cells derived from homogeneous non-self cells can also be used. In embodiments where cells derived from heterogeneous cells or derived from homogeneous non-self cells are used, the cells may require immunosuppressive treatment to suppress the rejection, in some cases. Note that in the present disclosure, the cells other than the self-derived cells, that is, cells derived from heterogeneous cells and cells derived from homogeneous non-self cells may also be collectively referred to as “non-self-derived cells”. In one embodiment of the present disclosure, the cells are autologous cells or non-autologous cells. In one embodiment of the present disclosure, the cells are autologous cells. In another embodiment of the present disclosure, the cells are non-autologous cells.

In the present disclosure, when the cell culture is a sheet-shaped cell culture, the sheet-shaped cell culture can be produced by any method known to a person skilled in the art (see, e.g., Japanese Patent Application No. 2018-140977, JP 2010-081829 A, JP 2011-110368 A, and the like). The method for producing a sheet-shaped cell culture typically includes, but is not limited to, a step of seeding cells on a substrate, a step of forming the seeded cells into a sheet-shaped cell culture, and a step of detaching the formed sheet-shaped cell culture from the substrate. Before the step of seeding cells on a substrate, a step of freezing cells and a step of thawing the cells may be performed. Further, a step of washing the cells may be performed after the step of thawing the cells. Each of these steps can be performed by any known technique suitable for the production of a sheet-shaped cell culture. The production method of the present disclosure may include a step of producing a sheet-shaped cell culture, and in that case, the step of producing a sheet-shaped cell culture may include one or two or more of the steps according to the method for producing a sheet-shaped cell culture as sub-steps. In one embodiment, a step of proliferating the cells is not included after the step of thawing the cells and before the step of seeding the cells on a substrate.

In one embodiment, in the cell culture of the present disclosure, cells are connected to each other through an extracellular matrix produced by the cells forming the cell culture. In one embodiment, the cell culture of the present disclosure contains an extracellular matrix.

In the present disclosure, although depending on various conditions, the extracellular matrix produced by the cells forming a cell culture is generated, for example, by culturing the cells for 24 hours or more, for example, for 24 hours, 30 hours, 36 hours, 42 hours, 48 hours, 54 hours, 60 hours, 66 hours, or 72 hours, after seeding the cells.

The extracellular matrix contributes to the binding of a cell culture to a transplant site, and can simplify the step of binding the cell culture to a transplant site, such as suturing, when the cell culture is transplanted. Further, the extracellular matrix is a cell-derived component, and thus, there is no problem in use of the extracellular matrix for transplantation together with the cell culture according to the present disclosure.

Examples of the substrate are not particularly limited as long as cells can form a cell culture on the substrate, and include containers made of various materials, and a solid or half-solid surface in a container. The container may have a structure/material that does not allow a liquid such as a liquid culture medium to permeate. Examples of the material include, but are not limited to, polyethylene, polypropylene, polytetrafluoroethylene (e.g., Teflon®), polyethylene terephthalate, polymethyl methacrylate, nylon-6,6, polyvinyl alcohol, cellulose, silicon, polystyrene, glass, polyacrylamide, polydimethyl acrylamide, and a metal (e.g., iron, stainless steel, aluminum, copper, or brass). Further, the container may have at least one flat surface. One example of the container includes a culture container that has the bottom made of a substrate capable of forming a cell culture and the liquid-impermeable side. Additional examples of the culture container include, but are not limited to, a cell-culture dish, and a cell-culture bottle. The bottom of the container may be transparent or opaque. If the bottom of a container is transparent, cells can be observed and counted from the underside of the container. Further, the container may have a solid or half-solid surface inside thereof. Examples of the solid surface include a plate, and a container, which are made of various materials as described above, and examples of the half-solid surface include a gel, and a soft polymer matrix. As the substrate, a substrate prepared by using the above-described materials may be used, or a commercially available material may be used. One non-limiting example of a substrate is a substrate having an adhesive surface, which is suitable for forming a sheet-shaped cell culture. Specifically, a substrate may have a hydrophilic surface (e.g., the substrate surface is coated with a hydrophilic compound such as corona discharge-treated polystyrene, a collagen gel, or a hydrophilic polymer), may be coated with an extracellular matrix (e.g., coated with collagen, fibronectin, laminin, vitronectin, proteoglycan, glycosaminoglycan, or the like), or may include a cell adhesion factor (e.g., a cadherin family, a selectin family, or an integrin family, or the like). Further, such a substrate may be commercially available (e.g., Corning® TC-Treated Culture Dish, or the like). The entirety or a portion of the substrate may be transparent or opaque.

The surface of the substrate may be coated with a material whose physical properties change in response to a stimulus (e.g., temperature or light). Non-limiting examples of the material may include a temperature-responsive material made of a homopolymer or a copolymer of a (meth)acrylamide compound; a N-alkyl-substituted (meth)acrylamide derivative (e.g., N-ethyl acrylamide, N-n-propyl acrylamide, N-n-propyl methacrylamide, N-isopropyl acrylamide, N-isopropyl methacrylamide, N-cyclopropyl acrylamide, N-cyclopropyl methacrylamide, N-ethoxyethyl acrylamide, N-ethoxyethyl methacrylamide, N-tetrahydrofurfuryl acrylamide, N-tetrahydrofurfuryl methacrylamide, or the like); a N,N-dialkyl-substituted (meth)acrylamide derivative (e.g., N,N-dimethyl (meth)acrylamide, N,N-ethyl methyl acrylamide, N,N-diethyl acrylamide, or the like); a (meth)acrylamide derivative having a cyclic group (e.g., 1-(1-oxo-2-propenyl)-pyrrolidine, 1-(1-oxo-2-propenyl)-piperidine, 4-(1-oxo-2-propenyl)-morpholine, 1-(1-oxo-2-methyl-2-propenyl)-pyrrolidine, 1-(1-oxo-2-methyl-2-propenyl)-piperidine, 4-(1-oxo-2-methyl-2-propenyl)-morpholine, or the like); a vinyl ether derivative (e.g., methyl vinyl ether); or a photoresponsive material such as a light-absorbing polymer having an azobenzene group, a copolymer of a vinyl derivative of triphenylmethane leucohydroxide and an acrylamide-based monomer, or N-isopropylacrylamide gel containing spirobenzopyran (see, e.g., JP H02-211865 A, and JP 2003-33177 A). By giving a predetermined stimulus to such a material, the physical properties, such as the hydrophilicity and the hydrophobicity, can be changed and the detachment of a cell culture attached on the material can be promoted. A culture dish coated with a temperature-responsive material is commercially available (e.g., UpCell® of CellSeed Inc., or Cepallet® of DIC Corporation), and such a culture dish can be used for the production method of the present disclosure.

The substrate may have various shapes, but is preferably flat. Further, the area is not particularly limited, and may be, for example, around 1 cm² to around 200 cm², around 2 cm² to around 100 cm², or around 3 cm² to around 50 cm². For example, as the substrate, a circular culture dish having a diameter of 10 cm can be used. In such an embodiment, the area of the culture dish is 56.7 cm².

The substrate may be coated with serum. By using a substrate coated with serum, a sheet-shaped cell culture with a higher density can be formed. The expression “coated with serum” means a state that a serum component adheres onto a surface of the substrate. Such a state can be obtained, as a non-limiting example, by processing a substrate with serum. The processing with serum includes bringing serum into contact with a substrate, and performing the incubation for a predetermined period of time as needed.

As the serum, heterologous serum and/or homologous serum can be used. In a case where a sheet-shaped cell culture is used for transplantation, the heterologous serum means serum derived from an organism of a species different from that of the recipient. For example, in a case where the recipient is a human, serum derived from a bovine or a horse, such as fetal bovine serum/fetal calf serum (FBS/FCS), calf serum (CS), horse serum (HS), or the like corresponds to the heterologous serum. Further, the expression “homologous serum” means serum derived from an organism of the same species as that of the recipient. For example, in a case where the recipient is a human, human serum corresponds to the homologous serum. The homologous serum includes self serum (also referred to as “autologous serum”), that is, serum derived from the recipient, and homologous non-autologous serum derived from an individual of the same species other than the recipient. Note that in the present disclosure, serum other than self serum, that is, heterologous serum and homologous non-autologous serum may be collectively referred to as “non-self serum”.

The serum for coating on a substrate is commercially available, or can be prepared from the blood collected from a desired organism by a conventional method. Specifically, for example, a method in which the collected blood is left to stand at room temperature for around 20 minutes to around 60 minutes so as to be coagulated, the coagulated blood is centrifuged at around 1000 G-Force (1000 g) to around 1200 g, and a supernatant is collected, or the like can be used.

In embodiments where the incubation is performed on a substrate, serum may be used in undiluted form, or may be diluted for use. The serum can be diluted, without any limitation, in any medium, such as water, a saline solution, various buffer solutions (e.g., PBS, HBSS and the like), various liquid media (e.g., DMEM, MEM, F12, DMEM/F12, DME, RPMI1640, MCDB (e.g., MCDB102, 104, 107, 120, 131, 153, or 199), L15, SkBM, or RITC80-7) or the like. The dilution concentration is not particularly limited as long as the serum component can adhere onto a substrate, and is, for example, around 0.5% to around 100% volume per volume (v/v), preferably around 1% to around 60% (v/v), and more preferably around 5% to around 40% (v/v).

The incubation time is also not particularly limited as long as the serum component can adhere onto a substrate, and may be, for example, around 1 hour to around 72 hours, preferably around 2 hours to around 48 hours, more preferably around 2 hours to around for 24 hours, and furthermore preferably around 2 hours to around 12 hours. The incubation temperature is also not particularly limited as long as the serum component can adhere onto a substrate, and is, for example, around 0 degrees Celsius (° C.) to around 60° C., preferably around 4° C. to around 45° C., and more preferably room temperature to around 40° C.

The serum may be discarded after incubation. As the technique for discarding the serum, suction with a pipette or the like, or a conventionally-used technique for discarding liquid such as decantation can be used. In a preferred embodiment of the present disclosure, after the serum is discarded, the substrate may be washed with a serum-free washing solution. The serum-free washing solution is not particularly limited as long as it is a liquid medium that does not contain serum and does not adversely affect the serum component adhered onto a substrate, and the washing can be performed, without any limitation, using water, a saline solution, various buffer solutions (e.g., PBS, HBSS and the like), various liquid media (e.g., DMEM, MEM, F12, DMEM/F12, DME, RPMI1640, MCDB (e.g., MCDB102, 104, 107, 120, 131, 153, or 199), L15, SkBM, or RITC80-7), or the like. As the washing technique, without any limitation, a conventionally-used technique for washing a substrate, such a technique in which a serum-free washing solution is added onto a substrate, stirred for a predetermined time (e.g., around 5 seconds to around 60 seconds), and then discarded, or the like can be used.

In the present disclosure, the substrate may be coated with a growth factor. As used herein, the expression “growth factor” means any substance that promotes proliferation of cells as compared with a substrate that is not coated with the growth factor, and examples of the growth factor include epidermal growth factor (EGF), vascular endothelial growth factor (VEGF), and fibroblast growth factor (FGF). The technique for coating on a substrate with a growth factor, the discarding technique, and the washing technique may be similar to or the same as those of serum except that the dilution concentration at the time of incubation is, for example, around 0.0001 micrograms/milliliter (μg/mL) to around 1 μg/mL, preferably around 0.0005 μg/mL to around 0.05 μg/mL, and more preferably around 0.001 μg/mL to around 0.01 μg/mL.

In the present disclosure, the substrate may be coated with a steroid drug. As used herein, the expression “steroid drug” refers to a compound that does not exert an adverse effect on the living body, such as adrenocortical insufficiency, or Cushing syndrome, among the compounds having a steroid nucleus. Examples of the compound include, but are not limited to, cortisol, prednisolone, triamcinolone, dexamethasone, and betamethasone. The technique for coating on a substrate with a steroid drug, the discarding technique, and the washing technique may be similar to or the same as those of serum except that the dilution concentration at the time of incubation is, for example, 0.1 μg/mL to around 100 μg/mL, preferably around 0.4 μg/mL to around 40 μg/mL, and more preferably around 1 μg/mL to around 10 μg/mL, as dexamethasone.

The substrate may be coated with the serum, the growth factor, or the steroid drug; or may be coated with any combination of the serum, the growth factor, and the steroid drug, that is, a combination of the serum and the growth factor, a combination of the serum and the steroid drug, a combination of the serum, the growth factor, and the steroid drug, or a combination of the growth factor and the steroid drug. In a case of coating with multiple components, the coating with these components may be performed at the same time by mixing the components with each other, or may be performed in separate steps.

The substrate may be seeded with cells immediately after being coated with serum and the like, or may also be stored after being coated and then seeded with cells. The coated substrate can be stored for a long period of time, for example, by keeping the substrate at around 4° C. or less, preferably around −20° C. or less, and more preferably around −80° C. or less.

In one embodiment, the present disclosure is a cell culture for treating inflammatory disease generated in the lower gastrointestinal tract. In the present disclosure, the lower gastrointestinal tract refers to the small intestine and the large intestine. The small intestine includes the jejunum and the ileum, and the large intestine includes the appendix, the cecum, the ascending colon, the transverse colon, the descending colon, the sigmoid colon, the upper S rectum, the upper rectum, the lower rectum, and the anal canal. Examples of the inflammatory disease generated in the lower part of the gastrointestinal tract include, but are not limited to, inflammation due to a malignant tumor such as small intestine cancer, or colon cancer, infectious enteritis, diverticulitis, and inflammatory bowel disease.

In one embodiment, the present disclosure is a cell culture for treating inflammatory bowel disease. In the present disclosure, the inflammatory bowel disease refers to chronic or ameliorated/relapsed inflammatory disease in the intestinal tract, and specifically refers to ulcerative colitis, Crohn disease, or intestinal Behcet disease.

In one embodiment, the cell culture according to the present disclosure is transplanted to the serosal side of the gastrointestinal tract, that is, the outside of the gastrointestinal tract. The cell culture may be transplanted away from the inflamed site, but it is preferable that all or part of the cell culture is transplanted to a position opposite to the position corresponding to the inflamed site across the muscle layer. In one embodiment, the cell culture may be transplanted to the serosal side of the gastrointestinal tract except for the position corresponding to the inflamed site. In one embodiment, the cell culture contains an extracellular matrix. In one embodiment of the present disclosure, the cell culture of the present disclosure is a sheet-shaped cell culture.

When there are multiple inflamed sites, one cell culture may be transplanted to the multiple inflamed sites, or multiple cell cultures depending on the number of inflamed sites may be transplanted to the multiple inflamed sites, respectively. When multiple cell cultures are transplanted, the multiple cell cultures may be each independently transplanted away from the inflamed site as long as the effect of the present disclosure is exerted, but it is preferable that all or part of the multiple cell cultures are transplanted to a position opposite to the position corresponding to the inflamed site across the muscle layer.

In order to transplant the cell culture according to the present disclosure to a subject, the morphology is not particularly limited, but is preferably morphology of a sheet-shaped cell culture.

The cell culture according to the present disclosure secretes myokine. The cell culture according to the present disclosure secretes myokine even in the morphology of a sheet-shaped cell culture. Further, by transplanting a sheet-shaped cell culture that secretes myokine to a subject having inflammatory disease, the inflammatory disease can be treated.

Without being bound by any particular theory, the action of the cell culture according to the present disclosure on inflammatory disease is considered because myokine secreted from the cell culture according to the present disclosure acts directly and/or indirectly on the cells at an inflamed site, and the proliferative effect of intestinal epithelial cells, the anti-inflammatory effect, and the anti-apoptotic effect are exerted. When the cell culture according to the present disclosure contains an extracellular matrix, it is considered that the bioadhesiveness of the cell culture to a transplant site is enhanced, and when myokine secreted from the cell culture is more produced, the myokine acts efficiently on the cells at an inflamed site.

Another aspect of the present disclosure relates to a method for producing a sheet-shaped cell culture containing cells derived from skeletal muscle, for treating inflammatory disease, including seeding cells on a substrate, forming the seeded cells into a sheet-shaped cell culture, and detaching the formed sheet-shaped cell culture from the substrate.

The sheet-shaped cell culture can be produced by any known method including seeding cells on a substrate, forming the seeded cells into a sheet-shaped cell culture, and detaching the formed sheet-shaped cell culture from the substrate.

As described above, in the present disclosure, the sheet-shaped cell culture can be produced by any method known to a person skilled in the art (see, e.g., Japanese Patent Application No. 2018-140977, JP 2010-081829 A, JP 2011-110368 A, and the like). The method for producing a sheet-shaped cell culture typically includes, but is not limited to, a step of seeding cells on a substrate, a step of forming the seeded cells into a sheet-shaped cell culture, and a step of detaching the formed sheet-shaped cell culture from the substrate.

Seeding of cells on a substrate can be performed by any known technique under any known conditions. The seeding of cells on a substrate may be performed, for example, by injecting a cell suspension in which cells are suspended in a liquid culture medium into a substrate (culture container). For the injection of the cell suspension, an instrument suitable for the injection operation of the cell suspension, such as a dropper or a pipette, can be used.

In one embodiment, the seeding can be performed at a density of around 7.1×10⁵ cells per centimeter squared (cells/cm²) to around 3.0×10⁶ cells/cm², around 7.3×10³ cells/cm² to around 2.8×10⁶ cells/cm², around 7.5×10³ cells/cm² to around 2.5×10⁶ cells/cm², around 7.8×10³ cells/cm² to around 2.3×10⁶ cells/cm², around 8.0×10³ cells/cm² to around 2.0×10⁶ cells/cm², around 8.5×10³ cells/cm² to around 1.8×10⁶ cells/cm², around 9.0×10³ cells/cm² to around 1.6×10⁶ cells/cm², around 1.0×10⁶ cells/cm² to around 1.6×10⁶ cells/cm², or the like.

Further, another aspect of the present disclosure relates to a kit for producing a cell culture containing cells derived from skeletal muscle, or the like, for treating the above-described inflammatory disease, including cells for producing a cell culture, and a culture medium and a substrate for culturing the cells.

The kit according to the present disclosure may further include, but is not limited to, for example, a washing solution, a buffer solution, a tube, an instrument used for cell culture, a transport container, instructions on the use method, and the like.

Examples of the instrument used for cell culture include a pipette, a cell strainer, and a cell scraper. Examples of the instructions on the use method include instructions for use, and a medium on which information about the production method and the use method has been recorded, for example, a flexible disk, a CD, a DVD, a Blu-ray disc, a memory card, a USB flash drive, or the like.

Further, another aspect of the present disclosure relates to a method for treating inflammatory disease, including performing transplantation of a cell culture containing cells derived from skeletal muscle. In one embodiment, the cell culture according to the present disclosure can be transplanted to or around the inflamed site to suppress inflammation in inflammatory disease. The cells forming the cell culture according to the present disclosure contain cells derived from skeletal muscle by 50% or more, 60% or more, 65% or more, 70% or more, 75% or more, 80% or more, 85% or more, or 90% or more, and preferably 60% or more. In one embodiment of the present disclosure, the cell culture is a sheet-shaped cell culture.

In one embodiment, the treatment of inflammatory disease is treatment of inflammatory disease generated in the lower gastrointestinal tract. In one embodiment, the treatment of inflammatory disease is treatment of inflammatory bowel disease.

In one embodiment, the cell culture according to the present disclosure is transplanted to the serosal side, that is, the outside of the gastrointestinal tract. The cell culture may be transplanted away from the inflamed site, but it is preferable that all or part of the cell culture is transplanted to a position opposite to the position corresponding to the inflamed site across the muscle layer. In one embodiment, the cell culture may be transplanted to the serosal side of the gastrointestinal tract except for the position corresponding to the inflamed site. In one embodiment, the cell culture contains an extracellular matrix. In one embodiment of the present disclosure, the cell culture of the present disclosure is a sheet-shaped cell culture.

When there are multiple inflamed sites, one cell culture may be transplanted to the multiple inflamed sites, or multiple cell cultures depending on the number of inflamed sites may be transplanted to the multiple inflamed sites, respectively. When multiple cell cultures are transplanted, the multiple cell cultures may be each independently transplanted away from the inflamed site, but it is preferable that all or part of the multiple cell cultures are transplanted to a position opposite to the position corresponding to the inflamed site across the muscle layer.

In one embodiment, in a case where the cell culture according to the present disclosure is a sheet-shaped cell culture, the sheet-shaped cell culture may have a support layer with a biodegradable gel such as a fibrin gel at the time of transplantation. In some embodiments, a method for forming a fibrin gel layer by adding a fibrinogen solution dropwise onto a sheet-shaped cell culture, and then spraying a thrombin solution (see, e.g., JP 6495603 B), or the like may be used.

As an example, an analysis of culture supernatant of sheet-shaped cell culture containing human myoblast cells was performed. (1) Preparation of sheet-shaped cell culture: The cryopreserved human myoblast cells (derived from a human skeletal muscle sample) were thawed at 37° C., and washed twice with a buffer solution containing 0.5% serum albumin. 5×10⁶ (5,000,000) to 5×10⁷ (50,000,000) cells were suspended in a 20% serum-containing medium, seeded in a flask, and then cultured for 2 to 3 days.

Into UpCell® (3.5-cm dish or 24-multiwell, CellSeed Inc.), a 20% serum-containing medium was added so as to cover the entire culture surface, and the processing was performed in an environment of 37° C. and 5% CO₂ for 3 hours to 3 days. After the processing, the added medium was discarded.

The cultured cells were collected, suspended in a 20% serum-containing medium, and seeded in processed UpCell® at a density of 2 to 20×10⁵ (200,000 to 2,000,000) cells/cm², and then the sheet-forming incubation was performed for around 1 day in an environment of 37° C. and 5% CO₂.

(2) Assay of secreted cytokine by using Bio-Plex™ Assay Kits (manufactured by Bio-Rad Laboratories, Inc.) was then performed, the culture supernatant of the sheet-shaped cell culture prepared in (1) was analyzed for 27 kinds of cytokines in accordance with the instructions of manufacturer.

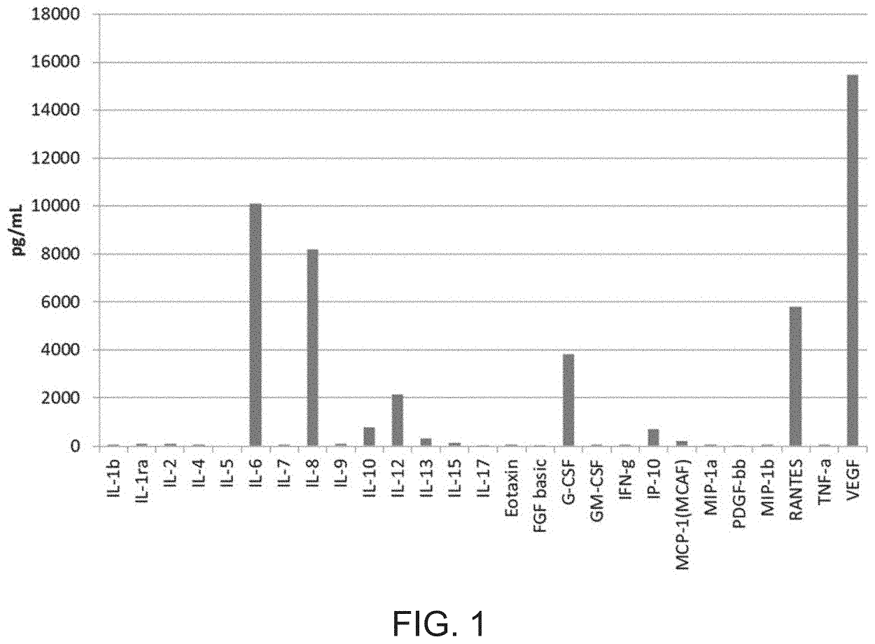

(3) Results are shown in FIG. 1. In the culture supernatant of the sheet-shaped cell culture containing human myoblast cells, VEGF, IL-6, IL-8, RANTES, G-CSF, IL-12, IL-10, IP-10, and the like were secreted.

As another example, a transplant test in mouse model of colitis was performed. First, a sheet-shaped cell culture was prepared. Skeletal muscle was collected from the lower limb of an 18-week old C57BL/6 mouse (CHARLES RIVER LABORATORIES JAPAN, INC.), processed with a solution containing collagenase and trypsin, and dispersed to single cells. Such cells were cultured in a 20% FBS-containing MCDB131 medium under the conditions of 37° C. and 5% CO₂ until the confluent was obtained, and the cells were collected. 1×10⁶ (1,000,000) cells of the collected cells were seeded in a 48-well temperature-responsive culture container (UpCell®), and cultured in a 20% FBS-containing DMEM/F12 medium for 6 hours or more, and the sheet-forming incubation was performed. After that, by lowering the temperature to 20° C., a sheet-shaped cell culture was detached from the temperature-responsive culture container, and collected.

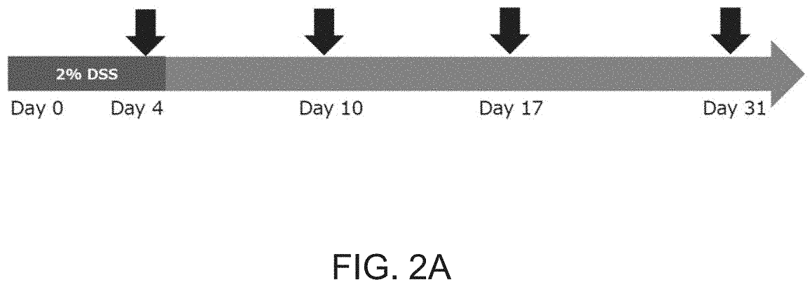

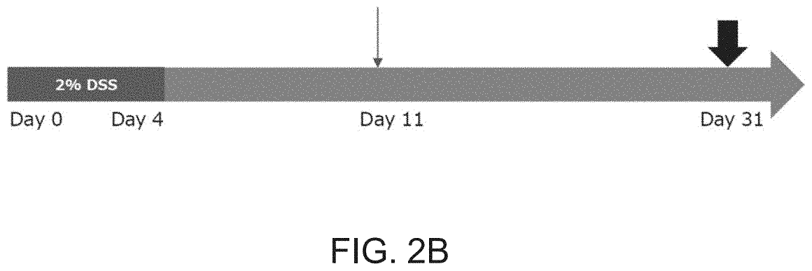

The test was carried out in accordance with the following method. The group composition of mice and the plan of autopsy and transplantation time are shown in FIG. 2.

In order to induce colitis, a 2% dextran sulfate sodium (DSS, MP Bio Japan K.K.) was orally administered to 7-week old C57BL/6 mice (Charles River Laboratories Japan, Inc.) in free drinking water for 4 days. The mice were randomly divided into a follow-up group and a test group. In the follow-up group, the autopsy was performed to examine the condition of the tissue on the 4th day, 10th day, 17th day, and 31st day counted from the start date of the free drinking water (water-free drinking) (hereinafter, the number of days is counted from the start date of the free drinking water). The test group was further divided into a sheet-shaped cell culture transplantation group and a sham surgery group. The mice in the sheet-shaped cell culture transplantation group were subjected to laparotomy under general anesthesia on the 11th day, and the sheet-shaped cell culture prepared in (1) was transplanted to the serosal side of the gastrointestinal tract where the large intestine was inflamed. The mice in the sham surgery group were subjected to only laparotomy under general anesthesia without transplanting any sheet-shaped cell culture. An autopsy was performed on the mice in the sheet-shaped cell culture transplantation group and in the sham surgery group on the 31st day to examine the condition of the tissue. Further, the lower gastrointestinal tract was collected from the mice in both of the groups, and the length of the large intestine was measured. In addition, the colon tissue was collected from the lower gastrointestinal tract and stained with hematoxylin-eosin stain, and a stained tissue image was obtained.

Further, in addition to the above-described histological experiment, for the mice in the sheet-shaped cell culture transplantation group and in the sham surgery group, the changes in the body weight, the property of stool, and the degree of bloody stool were observed, and the inflammation score (DAI) was calculated in accordance with the following Table 1 on the 3rd day, 9th day, 16th day, and 23rd day after the start of the test. The total score is 12 points, and it is indicated that the higher the numerical value is, the more severe the degree of inflammation is.

TABLE 1
Inflammation scores

| Score | Weight decrease | Stool property | Bloody stool |
|---|---|---|---|
| 0 | less than 1% | Normal | Normal |
| 1 | 1 to 5% | | |
| 2 | 5 to 10% | Loose stool | Small amount |
| 3 | 10 to 20% | | |
| 4 | 20% or more | Watery stool | Large amount |

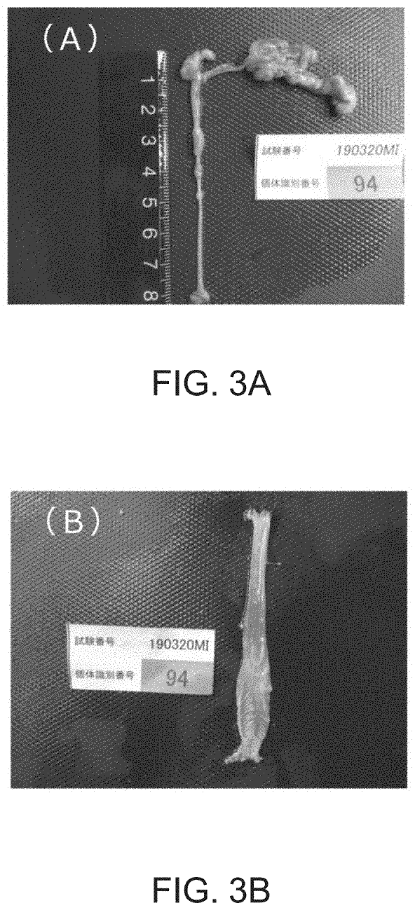

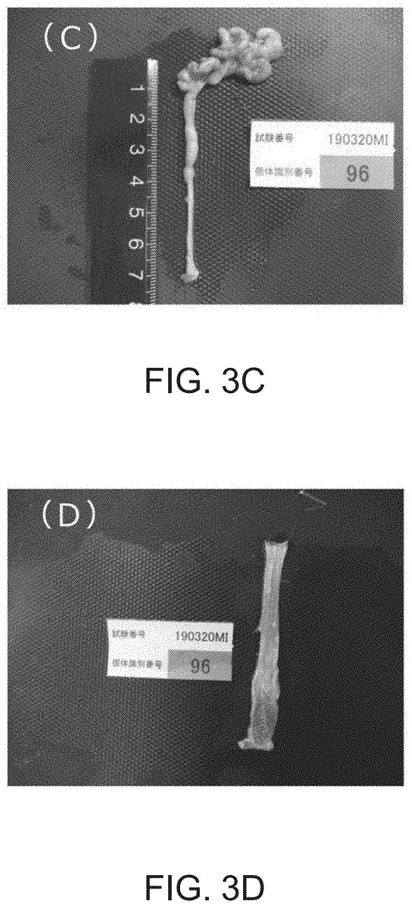

(3) Results: A photograph of the lower gastrointestinal tract collected from a mouse in the sheet-shaped cell culture transplantation group is shown in FIG. 3A, a photograph of the inner membrane of the gastrointestinal tract confirmed by cutting open the lower gastrointestinal tract is shown in FIG. 3B, a photograph of the lower gastrointestinal tract taken out from a mouse in the sham surgery group is shown in (FIG. 3C, and a photograph of the inner membrane of the gastrointestinal tract confirmed by cutting open the lower gastrointestinal tract is shown in FIG. 3D. The length of the large intestine of a mouse in the sheet-shaped cell culture transplantation group was 73 millimeters (mm), and the length of the large intestine of a mouse in the sham surgery group was 67±5 mm.

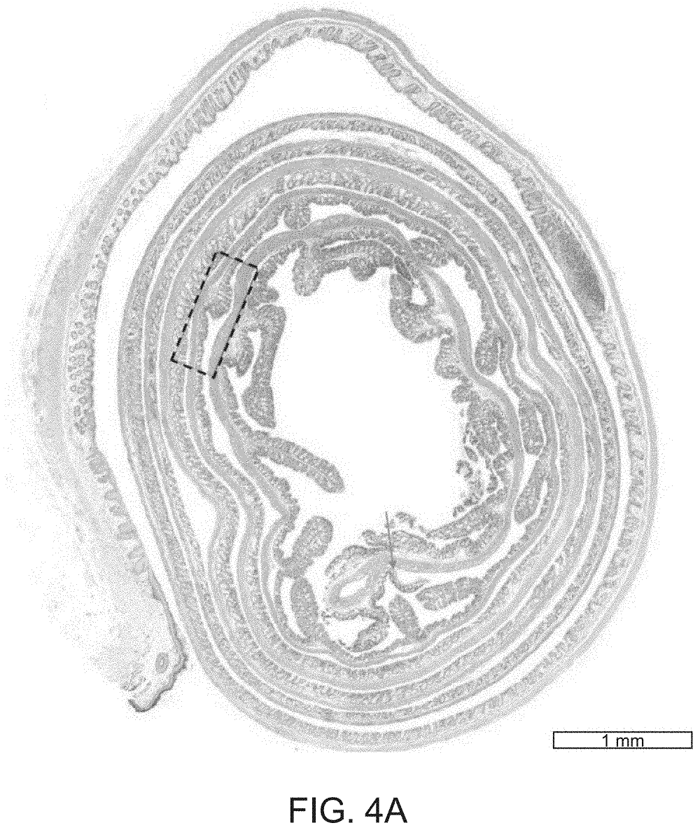

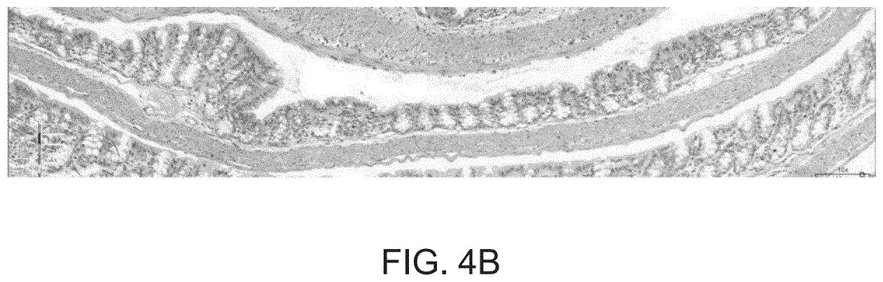

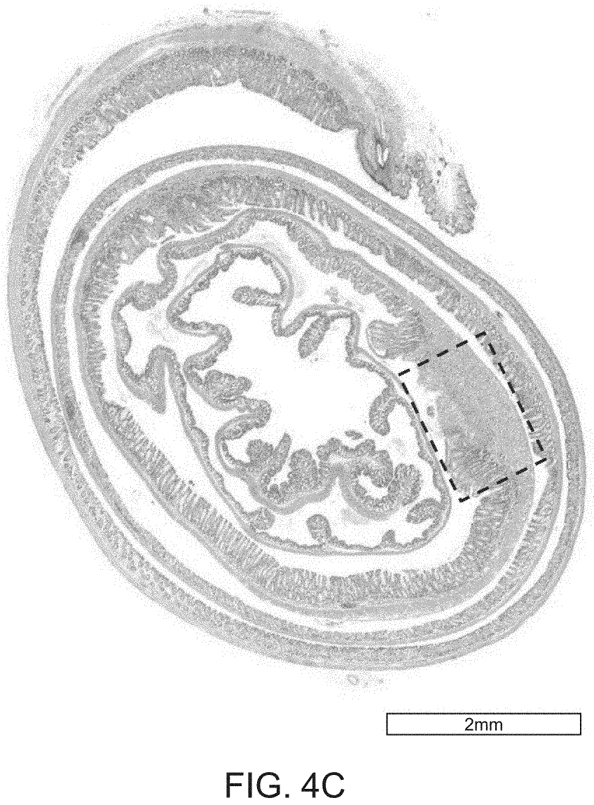

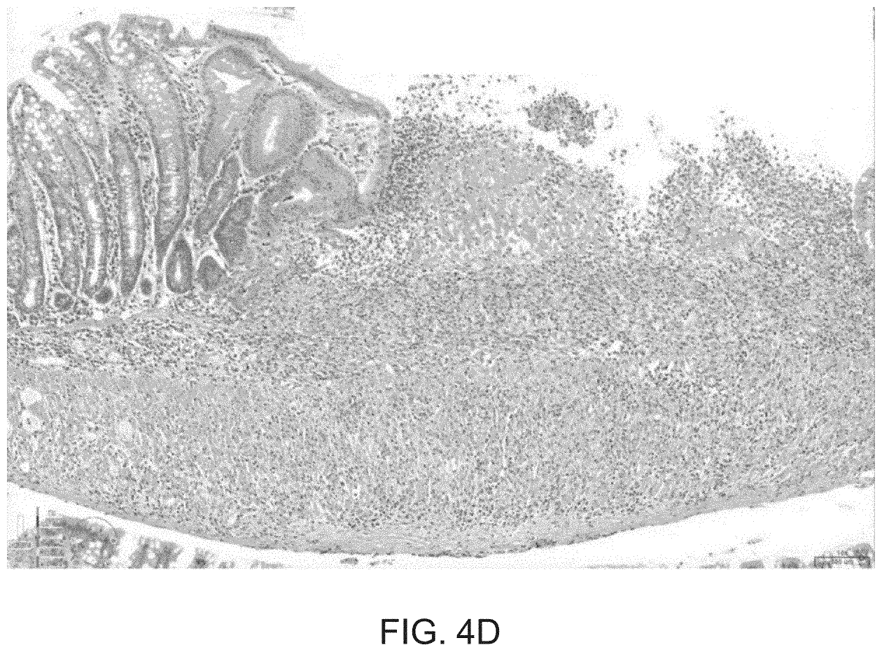

Among the stained tissue images obtained from the mice in the sheet-shaped cell culture transplantation group and in the sham surgery group, on which an autopsy was performed on the 31st day, a stained tissue image obtained from a mouse in the sheet-shaped cell culture transplantation group is shown in FIG. 4A, and an enlarged image of the dashed line portion is shown in FIG. 4B. Further, a stained tissue image obtained from a mouse in the sham surgery group is shown in FIG. 4C, and an enlarged image of the dashed line portion is shown in FIG. 4D.

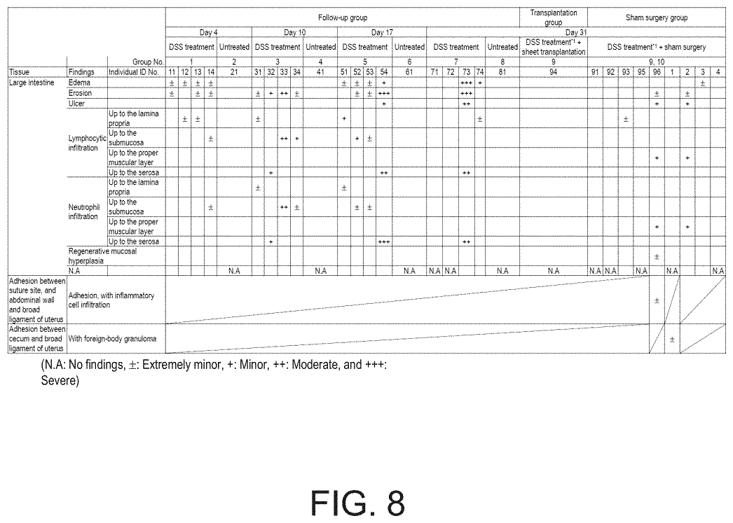

The histopathological evaluation of the tissues of the mice in the follow-up group, on which an autopsy was performed on the 4th day, 10th day, 17th day, and 31st day, and the mice in the sheet-shaped cell culture transplantation group and in the sham transplantation group, on which an autopsy was performed on the 31st day, is shown in FIG. 8, which shows a histopathological evaluation of tissues of mice in respective groups.

Erosion, ulcer, lymphocytic infiltration, and neutrophil infiltration were confirmed in the large intestine of the mice in the sham transplantation group on which an autopsy was performed on the 31st day, but abnormalities were not confirmed in the large intestine of the mouse in the sheet-shaped cell culture transplantation group on which an autopsy was performed on the 31st day.

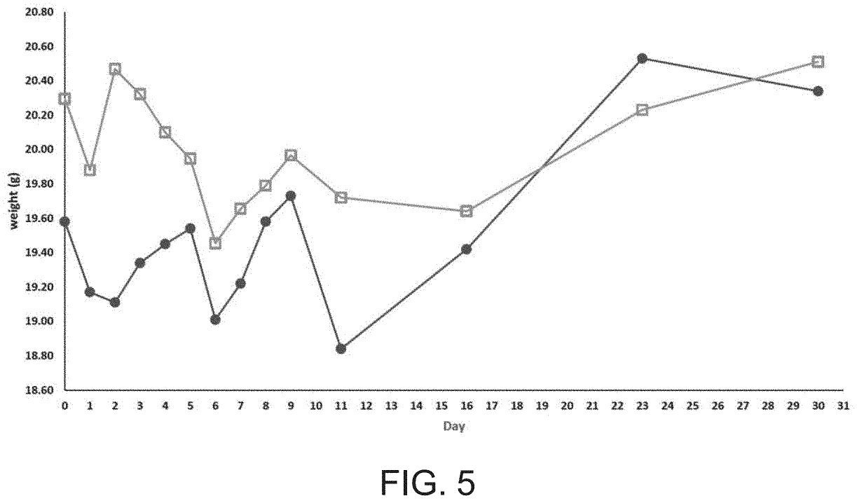

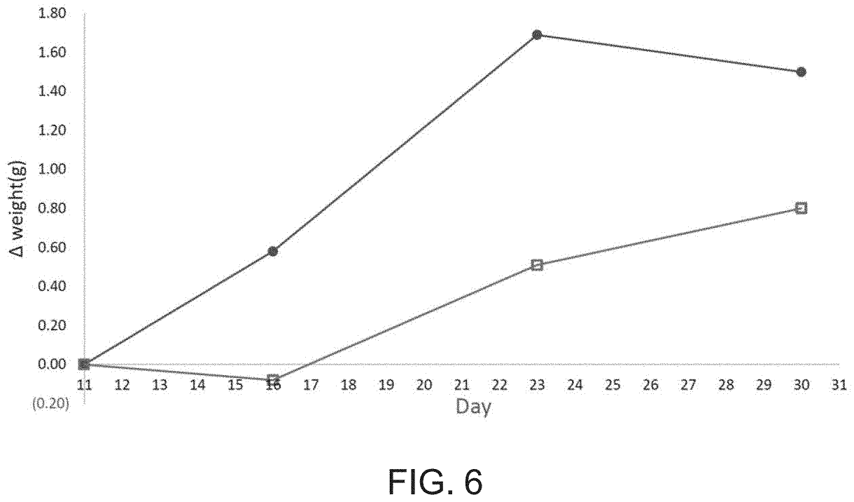

The changes in the body weight of the mice in the sheet-shaped cell culture group and in the sham surgery group are shown in FIG. 5. Further, the changes in the body weight of the mice in the sheet-shaped cell culture transplantation group and in the sham surgery group from the 11th day after the start of the test based on the body weight of each mouse on the 11 days after the start of the test are shown in FIG. 6. The squares indicate the mouse in the sham surgery group, and the black circles indicate the mouse in the sheet-shaped cell culture transplantation group. After the transplantation of a sheet-shaped cell culture, the mouse in the sheet-shaped cell culture transplantation group showed a significant increase in the body weight as compared with the mouse in the sham surgery group.

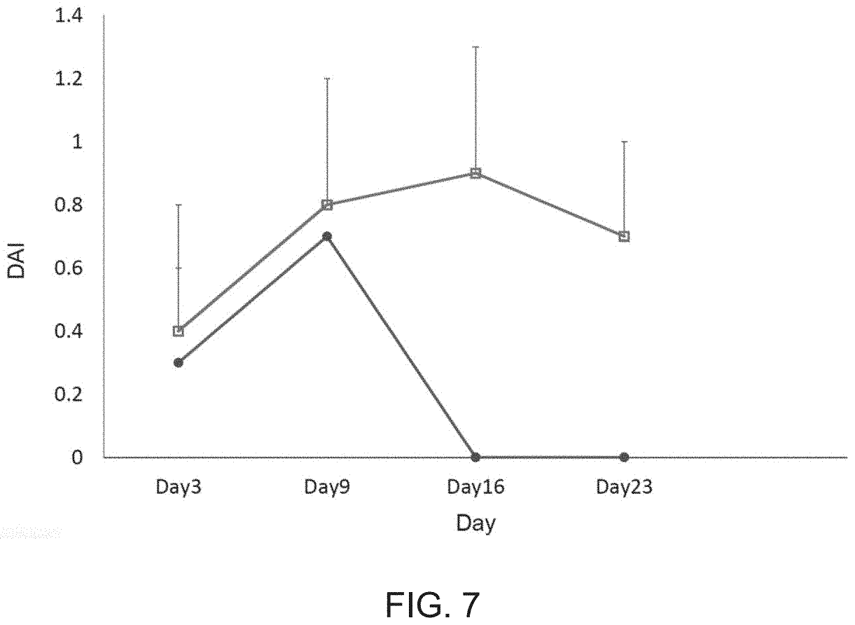

The inflammation scores (DAI) for the sheet-shaped cell culture transplantation group and the sham surgery group are shown in FIG. 7. The squares indicate the sham surgery group, and the black circles indicate the sheet-shaped cell culture transplantation group. In the sham surgery group, the scores were high also on the 16th day and 23rd day after the laparotomy. On the other hand, in the sheet-shaped cell culture transplantation group, surprisingly, the score on the 16th day after the laparotomy was 0, and the score on the 23rd day was also 0.

What is claimed is:

1. A cell culture for treating inflammatory disease, comprising cells derived from skeletal muscle.

2. The cell culture of claim 1, wherein the cell culture is a sheet-shaped cell culture.

3. The cell culture of claim 2, wherein the cell culture secretes myokine.

4. The cell culture of claim 1, wherein the cell culture secretes myokine.

5. The cell culture of claim 1, wherein the inflammatory disease is generated in the lower gastrointestinal tract.

6. The cell culture of claim 1, wherein the inflammatory disease is inflammatory bowel disease.

7. The cell culture of claim 1, wherein the cell culture is used for transplantation to the serosal side of the gastrointestinal tract.

8. The cell culture of claim 1, wherein the cell culture is formed by seeding cells on a substrate, forming the seeded cells into a sheet-shaped cell culture, and detaching the sheet-shaped cell culture from the substrate.

9. A kit for producing a cell culture, the kit comprising:
a plurality of cells; and
a culture medium; and
a substrate for culturing the plurality of cells,
wherein the plurality of cells are used to treat an inflammatory disease and are derived from skeletal muscle.

10. The kit of claim 9, wherein the plurality of cells is a sheet-shaped cell culture.

11. The kit of claim 10, wherein the cell culture secretes myokine.

12. The kit of claim 9, wherein the inflammatory disease is generated in the lower gastrointestinal tract.

13. A method for treating inflammatory disease, comprising transplanting a cell culture containing cells derived from skeletal muscle.

14. The method of claim 13, wherein the cell culture is a sheet-shaped cell culture.

15. The method of claim 14, wherein the cell culture secretes myokine.

16. The method of claim 13, wherein the cell culture secretes myokine.

17. The method of claim 13, wherein the inflammatory disease is generated in the lower gastrointestinal tract.

18. The method of claim 13, wherein the inflammatory disease is inflammatory bowel disease.

19. The method of claim 13, wherein transplantation of the cell culture is transplantation to the serosal side of the gastrointestinal tract.

20. The method of claim 13, further comprising:
seeding cells on a substrate;
forming the seeded cells into a sheet-shaped cell culture; and
detaching the sheet-shaped cell culture from the substrate.
